(19) United States
(12) Patent Application Publication
(10) Pub. No.: US 2025/0145889 A1
(43) Pub. Date: May 8, 2025

(54) METHOD FOR PROCESSING A BIOMASS MATERIAL

(71) Applicant: PROIONIC GMBH, Grambach (AT)

(72) Inventors: Roland KALB, Sinabelkirchen (AT); Markus DAMM, Graz (AT)

(21) Appl. No.: 18/835,975

(22) PCT Filed: Feb. 27, 2023
(86) PCT No.: PCT/EP2023/054871

§ 371 Date: Aug. 5, 2024

(30) Foreign Application Priority Data
Feb. 28, 2022 (EP) 22159280.1

(51) Int. Cl. C10G 1/02

(52) U.S. Cl. CPC C10G 1/02; C10G 2300/1014

(57) ABSTRACT

A method for processing a biomass material, wherein the biomass material is provided in the form of a mixture, the mixture comprising the biomass material and a medium comprising a nitrogen compound, and wherein said method comprises the steps of:
feeding the mixture into a vessel of an apparatus, wherein the apparatus comprises means to heat an internal surface of the vessel, and a plurality of blades arranged to rotate within the vessel,
treating the mixture with the plurality of blades such that the mixture is exposed to shearing, and forced to form a continuously renewed evaporation surface between the mixture and a gaseous phase,
allowing the nitrogen compound to form a gaseous form and allowing the gaseous form to pass through the evaporation surface and move into the gaseous phase,
removing the gaseous phase comprising the nitrogen compound in gaseous form from the vessel, and
collecting the remaining biomass material.

The present invention relates to a method of processing a biomass material, wherein the biomass material is provided in a mixture comprising the biomass material and a medium comprising a nitrogen compound.

### BACKGROUND OF THE INVENTION

Biomass processing is a critical aspect in the production of e.g. biofuels, bio-platform chemicals, biopolymers, bio-pharmaceuticals, bio-materials and any other kind of bioproducts from sustainable feedstocks.

The biomasses are macroscopically and chemically heterogeneous, i.e., the biomass typically comprises various components differing in their chemical nature and their physical properties. For example, lignocellulosic biomass being highly abundant includes the biopolymers cellulose, hemicellulose, and lignin. The process for separating components and gaining different value compounds of the biomass involve steps such as thermochemical (pre) treatment (temperature and pulping with non-neutral pH conditions) and biochemical conversion (i.e., enzymatic treatment for hydrolysis and fermentation of the components).

These steps can be performed in either aqueous or non-aqueous solvent systems. Besides water, other liquid (pre) treatment media with acidic or preferably alkaline properties turned out to be suitable, especially in the deconstruction and decomposition of the structure of the biomass mixture.

Nitrogen compounds were described as suitable (pre) treatment agents. For example, in the context of pretreatment of lignocellulosic biomass, which aims to solubilization of cellulosic materials in particular delignification, butylamine was disclosed for pretreatment (Tanaka, M., Robinson, C. W. & Moo-Young, M. Biotechnol Lett 5, 597-600 (1983). https://doi.org/10.1007/BF00130839). More recently, the class of alkanolamines has been disclosed as effective pretreatment solvents for biomass deconstruction and bioenergy production (Achinivu, E. C., Frank, S., Baral, N. R. et al. Green Chem 3, 8611 (2021). DOI: 10.1039/d1gc02667d).

Organic nitrogen compounds also include a subclass of ionic liquids having a cation with a nitrogen atom. In these compounds, the nitrogen formalistically carries the positive electrical charge of the cation. Ionic liquids (ILs) are described as a particular efficient set of compounds for the depolymerization and deconstruction of a range of feedstocks for biofuel production (Keasling, J., Garcia Martin, H., Lee, T. S. et al. Nat Rev Microbiol 19, 701-715 (2021). DOI: 10.1038/s41579-021-00577-w). More specifically, protic ILs, i.e., compounds with a protonated cation, are described as valuable pretreatment agents of lignocellulosic materials in WO 2013/192572 A1 and WO 2021/168154 A1.

Generally, ILs are a class of compounds comprising organic salts or salt mixtures consisting of organic cations and organic or inorganic anions with melting points below 100° C.—in the sense of acknowledged literature (e.g. Wasserscheid, Peter; Welton, Tom (Eds.); “Ionic Liquids in Synthesis, 2^{nd} Edition,” Wiley-VCH 2008; ISBN 978-3-527-31239-9; Rogers, Robin D.; Seddon, Kenneth R. (Eds.); “Ionic Liquids-Industrial Applications to Green Chemistry,” ACS Symposium Series 818, 2002; ISBN 0841237891). The melting point limit of 100° C. was set arbitrarily and in a wider sense, the definition can also include salt melts having a melting point above 100° C., but below 200° C. Additional inorganic salts may be dissolved in these salts, as well as molecular adjuvants. Ionic liquids have very interesting properties, such as in general very low vapor pressures, very wide liquidus ranges, good electric conductivity, and unusual solvation properties. When using ionic liquids, optimizing the properties for the respective use within wide limits can be achieved by varying the anion and cation structures or their combination, resulting in two degrees of freedom more compared to classical molecular structures, which is why ionic liquids are also called “designer solvents” (see, for example, Freemantle, M.; Chem. Eng. News, 78, 37 (2000)).

Due to their low vapor pressure, IL recovery may be challenging. Recovery and purification from solutions have been reviewed (Jingjing Zhou, Hong Sui, Zhidan Jia, Ziqi Yang, Lin He and Xingang Li, RSC Adv., 8, 32832-32864 (2018)). It was suggested to remove the IL from the pretreated biomass or the fermentation products by washing (Neupane, B., Konda, N. V. S. N. M., Singh, S., Simmons, B. A., Scown, C. D. ACS Sustainable Chem. Eng. 5 (11), 10176-10185 (2017) DOI: 10.1021/acssuschemeng.7b02116; WO 2013/192572 A1). This obviously comes with the disadvantage of using high amounts of washing water and needs subsequent energy intensive separation of washing water and ionic liquid in the solution state, to obtain economically feasible recovery rates of, e.g., above 99% to close to 100%.

Thus, distillable ionic liquids have recently gained attention in order to achieve at least partial separation of an ionic solvent from the biomass. The use of volatile salts for the pretreatment of biomass was disclosed in WO 2021/168154 A1. Therein, hydroxyethylammonium acetate, an example of a protic ionic liquid or acid-base conjugate salt, was identified to be suitable in biomass pretreatment and recovered with rates of about 85 to 95% after biomass pretreatment with 15% biomass loading using vacuum distillation. Exemplarily, distillation setups disclosed in WO 2021/168154 A1 cover a Kugelrohr apparatus (FIG. 1, therein) and vacuum distillation from a double walled mixing reactor (FIGS. 2 and 3, therein).

However, higher recovery rates are necessary to obtain highly pure forms of the remaining biomass as well as economic rentability by the recovery of expensive nitrogen compounds such as ionic liquids.

Accordingly, it is an object of the present invention to provide a method for processing a mixture containing a biomass material and a (pre) treatment medium comprising a nitrogen compound, such that an ionic liquid, is removed from the biomass material.

### DESCRIPTION OF THE PRESENT INVENTION

The present invention solves the problem by providing a method for processing a biomass material, wherein the biomass material is provided in the form of a mixture, the mixture comprising the biomass material and a medium comprising a nitrogen compound, and wherein said method comprises the steps of:
feeding the mixture into a vessel of an apparatus, wherein the apparatus comprises means to heat an internal surface of the vessel, and a plurality of blades arranged to rotate within the vessel,
treating the mixture with the plurality of blades such that the mixture is exposed to shearing, and forced to form a continuously renewed evaporation surface between the mixture and a gaseous phase,
allowing the nitrogen compound to form a gaseous form and allowing the gaseous form to pass through the evaporation surface and move into the gaseous phase,
removing the gaseous phase comprising the nitrogen compound in the gaseous form from the vessel, and
collecting the remaining biomass material.

The method allows to efficiently remove the nitrogen compound, preferably the complete medium, from the mixture comprising the biomass material. More particular, said medium may be referred to as a treatment and/or pretreatment medium, i.e., a (pre) treatment medium. The mechanical treatment in the apparatus is essential to repeatedly renew the surface of the mixture and thus, allows an efficient and rapid removal of the nitrogen compound in its gaseous form. The gaseous form of the nitrogen compound origins from the interface between the mixture and the gaseous phase and passes through this interface/surface, referred to as the evaporation surface. Thus, the method allows to separate the biomass material in the form of the remaining biomass material, typically in a solid state, from the nitrogen compound, preferably the complete liquid medium.

The separation of the biomass and the nitrogen compound occurs in that the nitrogen compound is removed in gas form, while the biomass is remaining in solid or liquid state. Accordingly, the method neither comprises nor relies on a step of washing and the nitrogen compound is not separated in liquid phase by a washing or coagulation solvent such as water.

The gaseous form of the nitrogen compound can be obtained by evaporation or decomposition of the nitrogen compound. If the nitrogen compound is distillable, it can be recovered after being evaporated into the gas phase and removed from the vessel, whereas otherwise it may be destroyed, when transformed in gaseous form.

In one embodiment, the nitrogen compound is distillable and allowed to evaporate, and the method further comprises a step of condensing the nitrogen compound out of the gaseous phase removed from the vessel. Thus, in this embodiment, the method of the invention is directed to recovering to the medium because at least a part of the medium is recovered. Preferably, the complete medium is distillable and recovered.

While the nitrogen compound is removed, the composition of the mixture changes and the biomass content increases. The biomass content in the mixture influences the rheological properties. The resulting change in rheological properties as well as the potential phase transition from a liquid, slurry, pulp or paste-like state towards a solid state complicates the processing of the mixture. In the method according to the invention, the mixture is treated to form an evaporation surface, for example spread out in films (or thin layers). The critical role of the evaporation surface was initially investigated with a static setup (Example 1 and FIG. 1). Additionally, the inventors found that (mechanical) treatment with a plurality of blades allows to overcome the previous described challenges to efficiently remove the medium from larger amounts of mixtures. The step of treating comprising shearing (mechanic processing) forces the mixture to form a continuously renewed evaporation surface to allow for separating the nitrogen compound from the biomass. The comparison with a classic rotatory evaporator proofed the importance of the mechanical treatment with a plurality of blades to force the formation of a continuously renewed evaporation surface between the mixture and a gaseous phase (Example 2 and FIGS. 2 and 3). Moreover, the examples proved that a recovery rate of above 98 wt. %, preferably above 99 wt. %, with respect to the initially used nitrogen compound could be reached within a reasonable processing time.

The biomass mixture typically has a high initial viscosity and further thickens as the medium is continuously removed thus, becomes more and more resistant to deformation, before it finally undergoes a phase transition from a liquid starting material to a paste and solid state. Additionally, the biomass mixture can incrust at the surface by conglutination, gumming and sticking, which may hinder or even prohibit the nitrogen compound to form a gaseous form, to pass through the evaporation surface and move into the gaseous phase. Preferred configurations of the apparatus, and in particular of the plurality of blades and the vessel, ensure a forced circulation of the load within the vessel and the formation of a continuously renewed evaporation surface between the load, and in particular the mixture, and a gaseous phase, and will also continuously remove, break up or destroy such incrustations.

Suitable types of apparatuses to perform the method according to the invention may differ from each other for example regarding the setup being either configured for batch-wise or continuous processing (and so in the time the mixture is treated), regarding the volume of the vessel, its orientation (horizontal or vertical), the shape of the blades, and their (rotational/peripheral) speed. On the other hand, suitable apparatuses share common constructional elements required for operating the method according to the invention. These are in particular the plurality of blades configured such that the load of the vessel can be exposed to shearing, repeatedly forced to form a continuously renewed evaporation surface between the mixture and a gaseous phase, and the vessel with an internal surface, which surface can be heated.

In the following, exemplary and preferred configurations of the apparatus, in particular the plurality of blades and the vessel are described.

The term plurality of blades is understood to refer to the collection of mechanical installations within the apparatus, which are configured to rotate within the vessel. The term blades should not be limiting regarding the geometrical form of these installations and include different shapes such as, e.g., agitator paddles, kneading elements, screw segments, cutting blades, discs, or rotating knifes.

When the plurality of blades exposes the mixture to shearing, incrustations break up and new evaporation surfaces are generated. Dependent on the shapes of the blades and the vessel, the evaporation surface forms films on the internal surface of the vessel, but also along any other interface between the mixture and the gaseous phase within the vessel. The term evaporation surface is used in a broad sense and includes any interface between the mixture and the gas phase such as formed by films, droplets, or strings of the mixture spread out in the vessel, freely falling or flung through the gas phase of the vessel.

In one embodiment, the step of treating the mixture with the plurality of blades includes that the mixture is repeatedly spread out in films, in particular films at the internal surface of the vessel. The term films is to be understood as a referring to a (thin) layers of the mixture, which thus provide a large evaporation surface.

Preferably, the plurality of blades is attached to one or more rotor element(s). Accordingly, in a preferred embodiment, the rotating plurality of blades defines at least one rotational axis.

Suitable apparatuses preferably have a specific arrangement of the plurality of blades with respect to the vessel to force a continuous renewal of the evaporation surface, while treating the load. The plurality of blades rotating in the vessel defines a three-dimensional outer geometric form, which can be referred to as the corresponding body of revolution. Preferably, the vessel's internal surface corresponds to a surface of revolution, which surface of revolution relates to the body of revolution defined by the rotating plurality of blades.

In apparatuses with this configuration of the plurality of blades and the vessel, the movement of the plurality of blades ensures that the complete volume of the load is mixed and participates in being spread out continuously, i.e., a full circulation of the load. It limits the dead room and the formation of pools, wherein material could escape from formation of an evaporation surface. With more than one rotational axis, the plurality of blades of one rotational axis may be arranged to closely pass by the plurality of blades and the rotor element of another rotational axis. Such arrangements are referred to as self-cleaning and further contribute to the full circulation of the load, and in particular the mixture.

Furthermore, the blades can strep the load, and in particular the mixture, on the internal surface of the vessel in the form of a thin film. For this purpose, it is preferred that the rotating plurality of blades has a small radial clearance (distance) to the internal surface of the vessel. This distance may vary along the height of the vessel.

In a preferred embodiment, the rotating plurality of blades has a radial clearance to the internal surface of the vessel in the range of from 0.1 to 50 mm, preferably from 0.5 to 20 mm, such as from 0.5 to 5 mm or from 1 to 10 mm.

In a preferred embodiment, the vessel has essentially the geometric form of a right cylinder. Preferably, the plurality of blades of each rotational axis extends to a uniform maximal radial extension (coaxial length) and the body of revolution of the rotating plurality of blades also has the geometric form of a right cylinder. If the plurality of blades defines one rotational axis, the vessel preferably is a cylinder with a circular base area. If the plurality of blades defines more rotational axes, the base area of the cylinder preferably has the shape of overlapping circles.

Thus, in a preferred embodiment, the vessel has essentially the geometric form of a right cylinder with a base area having the shape either of a circle or overlapping circles.

A cylindric vessel further has a height, which can be defined relative to its base area. Preferably, the height of the cylindric vessel corresponds to the length of the at least one rotational axis of the plurality of blades. A vessel essentially having the form of a cylinder may be oriented horizontally or vertically, depending on the orientation of the at least one rotational axis.

In a preferred embodiment, the height of the cylindric vessel (and the length of the at least one rotational axis of the plurality of blades) is higher than a diameter of the base area, wherein the diameter is the one diameter of the circular base, or the largest diameter of the overlapping circles of the base area. In this embodiment, the vessel provides a large internal surface, which advantageously allows for an efficient transfer of heat to the load, and in particular the mixture, in the vessel. Transfer of heat and thus transformation of the nitrogen compound into a gaseous form may be further enhanced by additional heated elements. For example, besides means to heat an internal surface of the vessel, the apparatus may further comprise means to heat a part of the plurality of blades or rotor elements.

Furthermore, it is preferred that a large evaporation surface is formed, when treating the load, and in particular the mixture. In preferred embodiments, the plurality of blades and the vessel are configured to allow for the formation of a continuous gaseous phase, i.e., they give rise to a single gas space of a specific volume. The configuration of the plurality of blades influences extension and volume of the gaseous phase. In particular, staggered arrangements of the blades, i.e., shifted along the rotational axis, and open blades, e.g., open, e.g., Z-shaped kneader blades, are preferred over massive screw segments, which would separate the gaseous phase, when the load, and in particular the mixture, is filled in the vessel.

In a preferred embodiment, the plurality of blades is configured to transport the load, and in particular the mixture, along the at least one rotational axis. Thus, the load, and in particular the mixture, is not only circulated around the rotational axis, but also moved in a further direction, e.g., along the height of the cylindric vessel or the at least one rotational axis. The mass transport along the rotational axis further contributes to ensure a complete circulation and efficient blending of the load, and in particular the mixture.

Additionally, a mass transport can contribute to operate the apparatus continuously or semi-continuously. The mass transport, preferably coaxial to a rotational axis, can contribute to feeding or discharging of the vessel, i.e., also influences the step of feeding the mixture into a vessel and collecting of the remaining biomass material. With more rotational axes, counter-rotating axis may neutralize a net mass transport. Thus, these embodiments typically are operated batch-wise.

Apparatuses suited for the method according to invention were previously used in other fields of technology. Suitable apparatuses are used for example in polymer processing technology, e.g., for devolatilizing of plastic polymers, i.e., separation of a fluid (gas or solvent) from a highly viscous polymer (melt).

In a preferred embodiment, the apparatus is selected from the group consisting of
thin-film processors, wherein the rotating plurality of blades defines one rotating axis, and the vessel essentially is a cylinder with a vertical orientation, and
processors, in particular large-volume processors, wherein the rotating plurality of blades defines one or two rotating axes, and the vessel essentially is a cylinder with a horizontal orientation.

Suitable thin-film processors, wherein the rotating plurality of blades defines one rotating axis, and the vessel essentially is a cylinder with a vertical orientation, are for example Filmtruder® or Viscon® processors as manufactured and provided by Buss-SMS-Canzler GmbH (Butzbach, Germany), or the Viscofilm® processors as provided by GIG Karasek GmbH (Gloggnitz-Stuppach, Austria). A scheme of a thin-film process with a vertically oriented cylindric vessel and a rotating plurality of blades of is exemplarily shown in FIG. 4 and discussed in Example 3. These types of thin-film processors are also used in the context of fiber technology, in particular for producing solutions of cellulose in aqueous tertiary amine oxides from a suspension of cellulose in an aqueous solution of the tertiary amine oxide (EP 0 356 419).

Suitable large-volume processors, wherein the rotating plurality of blades defines one or two rotating axes, and the vessel essentially is a cylinder with a horizontal orientation, are typically referred to as reactor or kneader, such as for example a two-rotor degasification kneader with open screw segments forming rotating elements. Suitable large-volume processors are ReaCom® and ReaSil® processors as manufactured and provided by Buss-SMS-Canzler GmbH (Butzbach, Germany), or the “Large Volume Devolatilization Kneaders” with one or two rotating axes as provided by LIST Technology AG (Arisdorf Switzerland). Exemplarily, a scheme of a large-volume processor, i.e., a sectional view through a horizontal vessel with the plurality of blades defining two rotating axes, is shown in FIG. 5 and discussed in Example 3.

Other commercial processors with rotating blades and (evaluable) vessel with means to heat the internal surface include for example also extruders, such as twin-screw extruders. While extruders typically are configured to work with highly viscous loads and certain setups are provided with means for removal of a gas phase for devolatilizing and degasification, they are less preferred for the method according to the invention as they typically result in a smaller effective evaporation surface. Furthermore, their screw-shaped blades may prevent a large and continuous gas phase.

In suitable industrial apparatuses, the plurality of blades is configured to rotate and work with a high power to ensure the mechanical processing. Industrial processors typically may be characterized by parameters selected from the group consisting of peripheral speed (i.e., velocity of the blades at their maximum peripheral extension), area of the internal surface (i.e., surface of heat transfer), average residence time (i.e., time the mixture is processed within the vessel), evaporation surface renewal rate, specific evaporation performance, specific product throughput, and specific drive power (i.e., mechanical energy input to rotate the blades).

Industrial thin-film processors typically are characterized by
a peripheral speed in the range of from 0.5 to 5 m/s, such as from 1 to 2 m/s,
an area of the internal surface in the range of from 12 to 40 m²,
an average residence time in the range of from 2 to 15 min, such as from 4 to 8 min,
an evaporation surface renewal rate in the range of from 0.25-25 s⁻¹, such as from 2 to 10 s⁻¹,
a specific evaporation performance in the range of from 10 to 250 kg/h·m², such as from 20 to 150 kg/h·m²,
a specific product throughput in the range of from 20 to 300 kg/h·m², such as from 40 to 150 kg/h·m², and/or
a specific drive power in the range of from 0.01 to 0.20 kWh/kg, such as from 0.05 to 0.12 kWh/kg.

Industrial processors with horizontal orientation of a cylindric vessel, typically are characterized by
a peripheral speed in the range of from 0.2 to 2 m/s, such as 0.5 to 1.2 m/s,
an average residence time in the range of from 8 to 200 min,
a specific drive power in the range of from 0.01 to 0.20 kWh/kg and/or
an interior space with a minimum volume of approximately 3 to 50,000 liters and a normal volume of approximately 1,000 to 20,000 liters (in particular for large-volume processors).

In a preferred embodiment, during the step of treating the mixture, the plurality of blades rotates with a maximal peripheral speed in the range of from 0.2 to 5 m/s, preferably, from 0.5 to 2 m/s, such as about 1 m/s.

In the step of removing the gaseous phase, the nitrogen compound in gaseous form is removed together with the gaseous phase. The movement of the gaseous phase defines a gas stream within the vessel, more particular the gas space of the vessel. In embodiments referred to as thin-film processors, the gas stream is preferably coaxial to the mass transport along the at least one rotating axis and may be co-current or countercurrent.

The active withdrawal of the gaseous phase can be efficiently achieved by a vacuum, which also supports the transformation of the nitrogen compound in a gaseous form. Accordingly, it is preferred that the vessel is evacuable and the apparatus has a means for evacuation.

Alternatively or additionally to a gas stream imposed by a vacuum, the removal of the gaseous phase may also be achieved by a guided strip gas stream, i.e., by externally pumping a strip gas (carrier gas) through the vessel. Further, volatile components of the medium may contribute to the gas stream and entrain the nitrogen compound. However, due to necessity of further means or components, these embodiments are less preferred.

Accordingly, in one embodiment, the gaseous phase is removed with a gas stream withdrawn by means of evacuation and/or a guided strip gas stream, preferably evacuation.

Preferably, the method according to the invention, is performed under evacuation, i.e., at a reduced pressure within the vessel, a pressure below normal pressure (i.e., below about 1,000 mbar or 1 atm). The apparatuses described above can operate with pressures down to 10⁻² mbar in the vessel. A pressure in the range of from 0.5 to 10 mbar, such as 1 mbar, can be economically maintained in the vessel by convenient means for evacuations such as liquid ring vacuum pumps, membrane vacuum pumps, screw vacuum pumps, roots vacuum pumps, and scroll vacuum pumps. Preferably, the reduced pressure is applied during the steps of treating the mixture and removing the gaseous phase, which steps typically are processed essentially simultaneously.

Accordingly, in a preferred embodiment, the method is performed at a pressure in the range of from 10⁻² mbar to 10³ mbar, preferably in the range of from 10⁻¹ mbar to 100 mbar, more preferably in the range of from 0.5 to 10 mbar, such as 1 mbar.

Besides low pressure, heating of the internal surface influences the transformation of the nitrogen compound in a gaseous form.

In a preferred embodiment, the internal surface is heated to a temperature in the range of from 20° C. to 200° C., preferably of from 50° C. to 170° C., more preferably of from 80° C. to 160° C., such as from 100 to 140° C.

The person of ordinary skill in the art will select the temperature of the internal surface depending on the desired target temperature of the mixture and consider that the desired target temperature depends on the applied pressure and the properties of the compound to be removed, which are discussed below. Furthermore, the temperature sensitivity of the biomass material should be considered, which is why a temperature of below 160° C. is more preferred for certain applications. For example, when the remaining biomass should include saccharides/sugars, they could decompose at higher temperatures.

In an embodiment of the invention, the mixture has an initial shear viscosity of from 10 to 50,000 Pa*s, typically in the range of from 100 to 20,000 Pa*s, such as about 500 to 15,000 Pa*s, wherein the shear viscosity of the initial mixtures preferably is determined using an oscillation rheometer with a two plates geometry at a shear rate of 1 s⁻¹ and a measuring temperature of about 25° C.

The advantages of the present invention are especially valuable, wherein the mixture is a heterogeneous mixture with challenging rheological properties such as high viscosity. The above described thin-film processors are known for processing loads with viscosities up to 10,000 Pa*s or 15,000 Pa*s at the processing conditions and large-volume processors are known for processing loads with viscosities up to 50,000 Pa*s at the processing conditions.

A mixture of a biomass material and a medium, as it typically results from a biomass pretreatment step, typically is heterogeneous and may also be described as a slurry or suspension. In one embodiment, the biomass material is a non-uniformly shaped or fibrous material present as disperse phase in the mixture. The mixture may show elements of a fluid and/or a solid-like rheological behavior.

Exemplarily, mixtures with 3 wt. % to 60 wt. % of switch grass biomass material pretreated with the ionic liquid 1-ethyl-3-methylimidazolium acetate have been described to have an apparent dynamic, i.e., shear viscosity in the range of from about 10 to 1,000 Pa*s at a shear rate of 1/s and 25° C. using a two plates rheometer, wherein a sample was sandwiched between 25 mm serrated plates (Cruz, A. G., Scullin, C., Mu, C. et al. Biotechnol Biofuels 6, 52 (2013) https://doi.org/10.1186/1754-6834-6-52). The mixtures showed strong shear thinning and this effect was larger for higher biomass contents. Exemplarily, for a biomass loading of 50 wt. %, the complex viscosity of these pretreated slurries showed significant decreases of about 130-fold, when the frequency was increased by 100-fold. While the apparent viscosity decreases with higher shear rates for shear thinning mixtures, the viscosity of the mixture will typically increase as the liquid medium is continuously removed during the method. Furthermore, factors such as the measuring temperature influence the rheological properties. Thus, due to their non-Newtonian behavior and the multi-factorial dependency of the shear viscosity, an individual shear viscosity value cannot characterize the mixture's rheology in a comprehensive manner. Thus, the initial shear viscosity as defined above may not be appropriate to fully describe the rheological behavior within the method according to the invention.

As noted above, the initial shear viscosity of the mixture mainly depends on the nature of the biomass and the medium and their quantitative relation, i.e., their contents/biomass load. Typically, the amount of medium and the nitrogen compound used in biomass processing represents a relevant cost factor and thus is as low as possible. With the efficient recovery provided by the method according to the present invention, these costs can be limited and the use of medium, particular the (pre) treatment medium, in excess can be considered to explore its advantageous effects during the (pre) treatment.

In a preferred embodiment, the mixture has an initial content of the biomass in the range of from 10 to 80 wt. %, preferably from 5 to 70 wt. %, more preferably from 10 to 50 wt. %, such as from 15 to 40 wt. % or from 15 to 25 wt. %.

Accordingly, wherein the mixture consists of the biomass and the medium, the mixture has an initial content of medium in the range of from 20 to 90 wt. %, preferably from 30 to 95 wt. %, more preferably from 50 to 90 wt. %, such as from 60 to 85 wt. % or from 75 to 85 wt. %.

Herein, the term medium refers to a composition comprising or consisting of the nitrogen compound and being in liquid state. The term (pre) treatment medium was chosen to cover the aspect that the medium comprising the nitrogen compound preferably is used for pretreatment and/or treatment of a biomass. It may be used in preprocessing such as deconstructing the (cellular) structure of the biomass before applying subsequent steps of for example enzymatic/microbial hydrolyzation or fermentation (pretreatment medium). However, the term (pre) treatment medium should not exclude cases, wherein no subsequent steps are performed and eventually no further treatment medium is used, such that the (pre) treatment medium may be referred to as the treatment medium of the process.

The person of skill in the art will make the selection of the medium and the comprised nitrogen compound mainly based on the properties required for the desired (pre) treatment effect on the biomass. On the other hand, the following specifications with respect to the medium are especially preferred in the context of the method according to the invention.

The medium comprises the nitrogen compound, preferably a protonable or protonated nitrogen compound. The term protonable is synonym to protonatable or protonizable. A protonable nitrogen compound refers to a compound comprising a nitrogen atom having a free electron pair such that it can bind/accept a hydrogen ion/proton (H⁺), i.e., a free Brønsted base. On the other hand, a protonated nitrogen compound is a compound comprising a nitrogen atom having a detachable/donatable hydrogen ion/proton (H⁺), i.e., a Brønsted acid. A protonable nitrogen compound can be reacted to a protonated nitrogen compound by protonation and vice versa by deprotonation. Given that these reactions can compete, the term nitrogen compound includes both, compounds comprising a protonable nitrogen compound, compounds comprising a protonated nitrogen compound as well as hybrid forms thereof.

In a preferred embodiment, the nitrogen compound is the main component of the medium. Preferably, the nitrogen compound is in liquid state at room temperature or the temperature of a (pre) treatment step that precedes the method according to the invention and thus, the nitrogen compound itself may constitute the medium, in particular the (pre) treatment medium.

In a preferred embodiment, the nitrogen compound is selected from the group consisting of
neutral nitrogen compounds and
nitrogen based protic ionic liquids, preferably ionic liquids being selected from acid base conjugate salts and alkyl carbamate ionic liquids,
and hybrid forms thereof.

The term neutral nitrogen compound is to be understood to refer to a compound being neutral in terms of electrical charge and having a free valence at the nitrogen, such that they are protonable nitrogen compounds. Suitable liquid nitrogen compounds are also described as nitrogenous bases and used for example as polar and basic solvents in organic chemistry. Due to their alkaline properties and their liquid nature, some of these neutral nitrogen compound should be useful (pre) treatment media for biomasses. Indeed, exemplarily neutral nitrogen compounds such as butylamine and alkanolamines have been discussed for this purpose (Tanaka, M., Robinson, C. W. & Moo-Young, M. Biotechnol Lett 5, 597-600 (1983). https://doi.org/10.1007/BF00130839; Achinivu, E. C., Frank, S., Baral, N. R. et al. Green Chem 3, 8611 (2021). DOI: 10.1039/dlgc02667d). Some neutral nitrogen compounds, however, may not be preferred for reasons of security due to flammability or environmental and health hazards.

In a preferred embodiment of the invention, a neutral nitrogen compound is as a compound according to formula B

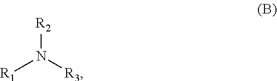

wherein R₁, R₂ and R₃ independently from each other are selected from hydrogen and alkyl groups with 1 to 6 carbon atoms,
or R₁ and R₂ together with the nitrogen atom form an aliphatic or aromatic 5- to 6-membered heterocyclic group and R₃ is selected from hydrogen and alkyl groups with 1 to 6 carbon atoms or R₃ is absent if R₁ and R₂ form an aromatic heterocyclic group,
wherein any alkyl group independently from each other is unsubstituted or substituted with a hydroxyl group or one or two further NR₄R₅ group(s), wherein R⁴ and R⁵ independently from each other are selected from hydrogen and unsubstituted alkyl groups with 1 to 3 carbon atoms, and
wherein any heterocyclic group is aliphatic or aromatic and may further comprise one to two further heteroatoms selected from O, S, N, and NR₆, wherein R₆ is selected from hydrogen and unsubstituted alkyl groups with 1 to 4 carbon atoms, preferably O, N and R₆.

Any alkyl group with 1 to x carbon atoms is also referred to as C₁-Cₓ-alkyl and is to be understood to include linear C₁-Cₓ-alkyl (such as methyl, ethyl, etc.), branched C₃-Cₓ-alkyl (such as iso-propyl, but-2-yl, tert-butyl, etc.) and cyclic C₅-Cₓ-alkyl (such as cyclopentyl and cyclohexyl).

In a preferred embodiment, the neutral nitrogen compound has a boiling point of at most about 250° C. at normal pressure.

Based on the inventors' considerations, a neutral nitrogen compound having a boiling point (T_{b}) of at most about 250° C. at normal pressure should be distillable under preferred conditions (temperature, pressure) of the method according to the invention.

In a preferred embodiment, the neutral nitrogen compound is a compound according to formula B and selected from the group consisting of ammoniac (NH₃) as well as different organic groups such as
primary, secondary, and tertiary alkylamines,
alkanolamines (amines substituted with hydroxyalkyl-group) such as e.g. N,N-dimethylethanolamine, ethanolamine, N-methylethanolamine, 2-(dimethylamino) ethanol, 2-(methylamino) ethanol, 2-amino-1-propanol, 3-amino-1-propanol, 3-amino-2-propanol, 2-amino-1-butanol, 3-amino-1-butanol, and 4-amino-1-butanol,
diamines (alkylamines further substituted with —NR₃R₄ group) such as the alkyldiamines according the formula H₂N—(CH₂)ₓ—NH₂, wherein x is in the range of from 2 to 6, 1,3-diamino-2-propanol, N-methylethylenediamine, and N,N,N′,N′-tetramethylethylendiamine (TMEDA),
guanidines (methylamines further substituted with two —NR₃R₄ groups) such as 1,1,3,3-tetramethylguanidine and,
heterocycles like pyridine, pyrrole, morpholine, N-alkylmorpholine, wherein alkyl is C₁-C₄-alkyl, piperidine, N-alkylpiperidine, wherein alkyl is C₁-C₄-alkyl, pyrimidine, pyrazine, pyridazine, piperazine, N-methylpiperazine, N-ethylpiperazine, N,N′-dimethylpiperazine, N,N′-diethylpiperazine, imidazole, N-methylimidazole, N-ethylimidazole, pyrrolidine, N-methylpyrrolidine, N-ethylpyrrolidine, and N-butylpyrrolidine.

More preferably, the neutral nitrogen compound is selected from the group consisting of NH₃, methylamine, ethylamine, propylamine, isopropylamine, butylamine, hexylamine, octylamine, dimethylamine, diethylamine, dipropylamine, dibutylamine, trimethylamine, triethylamine, tripropylamine, tributylamine, dimethylethylamine, dimethylpropylamine, dimethylbutylamine, cyclohexylamine, ethanolamine, N-methylethanolamine, N,N-dimethylethanolamine, 3-amino-1-propanol, 3-amino-2-propanol, 4-amino-1-butanol, ethylenediamine, 1,3-diaminopropane, 1,4-diaminobutane, 1,3-diamino-2-propanol, 1,1,3,3-tetramethylguanidine, pyridine, pyrrole, morpholine, N-methylmorpholine, N-ethylmorpholine, piperidine, N-methylpiperidine, N-ethylpiperidine, imidazole, N-methylimidazole, N-ethylimidazole, pyrrolidine, N-methylpyrrolidine, N-ethylpyrrolidine, N-butylpyrrolidine, N-methylethylenediamine, and N,N,N′,N′-tetramethylethylendiamine.

These preferred neutral nitrogen compounds are selected based on their commercial availability as well as their boiling point (T_{b}) being at about 250° C. or below at normal pressure.

Within each of the different organic groups, as mentioned above, the boiling point of the neutral nitrogen compound tends to increase with the molecular weight. Preferably the neutral nitrogen compound has a molecular weight of about 200 Da and below.

In another embodiment, the nitrogen compound is selected from the group of ionic liquids, in particular nitrogen-based protic ionic liquids. The class of nitrogen-based protic ionic liquids meets the criteria of a nitrogen compounds as defined above as the cation of these ionic liquids represents a protonated (organic) nitrogen compound.

In a preferred embodiment of the invention, a nitrogen-based protic ionic liquid is defined according to formula (C)

[BH_{y}^{y+}]ₓ[A^{x-}]_{y} (C),
wherein BH_{y}^{y+} is a cation and derived from of a compound according to formula B as defined above by protonation,
A^{x-} is an inorganic or organic anion,
and x and y are integers, preferably x is in the range of from 1 to 3 and y is in the range of from 1 to 2, more preferably x and y are 1.

Preferably, the ionic liquid is an acid base conjugate salt ionic liquid, i.e., a compound according to formula (C), wherein A is an anion derived from an acid by deprotonation. The formation of an acid base conjugate salt ionic liquid may be described according to the simplified equation I:

B+HA [symbol] [BH⁺+A^{-]} (I),
wherein B is a neutral nitrogen compound of formula B (i.e., a base B) and HA is an acid.

The stoichiometry of equation I depends on the number of protons the nitrogen compound can bind and the number of the protons the acid can donate. In the following equation II, acids and bases donating/accepting more than one proton can be considered:

x·B+y·HₙA [symbol] x·(BH_{y})^{y+}+y·(H₍ₙ₋ₓ₎A)^{x-} (II),

wherein B is a nitrogen compound of formula B protonable with up to m protons, HₙA is an acid donating up to n protons, and x and y are integers, wherein x is within the range of from 1 to n and y is within the range of from 1 to m.

As shown in the equations I to II, acid base conjugate salt ionic liquids are in equilibrium with neutral compounds. Thus, they may combine the advantages of an ionic compound with the volatility of their precursors (i.e., free base B and acid HₙA, e.g. B and HA). Upon cooling, they may be recovered in the ionic form. Accordingly, some of these ionic liquids are referred to as distillable acid base conjugate salt (DABCS), a term which was also used in WO 2021/168154.

In a preferred embodiment, the ionic liquid has a pseudo boiling point (T_{b}) of about 210° C. or below, preferably about 200° C. or below, at normal pressure (i.e., about 1,000 mbar or about 1 atm).

Based on the inventors' considerations, an ionic liquid having a pseudo boiling point (T_{b}) of at most about 210° C. at normal pressure should be distillable under preferred conditions (temperature, pressure) of the method according to the invention.

The pseudo boiling point of an ionic liquid defines a temperature at a given pressure, e.g., normal pressure, at which minimum temperature the ionic liquid is transferred from liquid into a gaseous form, or at which maximum temperature the ionic liquid can be recovered from gaseous form in the liquid form.

The pseudo boiling point T_{b} of a compound can be detected for example by means of thermogravimetric analysis (TGA) at a typical heating rate of e.g. 5 K/min as an onset-temperature of mass loss and as a strong endothermic signal in a differential scanning calorimetry (DSC) experiment under analogue conditions, as known to a person skilled in the art.

For an acid base conjugate salt ionic liquid, the gaseous form preferably consists of the evaporable neutral base and the evaporable neutral acid (B and HₙA, e.g. B and HA). Thus, a distillable ionic liquid is at least partially transferred into their neutral precursors to form the gaseous form. Accordingly, the pseudo boiling point of the acid base conjugate ionic liquid depends i) on the boiling point of the base B, from which the cation of an acid base conjugate salt ionic liquid is derived (T_{b} B), ii) the boiling point of the acid HₙA, from which the anion of an acid base conjugate salt ionic liquid is derived (T_{b} HA) as well as iii) the equilibrium reaction between the neutral and ionized compounds. This equilibrium reaction is strongly influenced by the Brønsted basicity of the base and the Brønsted acidity of the acid. These two factors—and especially the delta between basicity and acidity—are driving the position of the equilibrium for the above-mentioned equations (I) and (II), i.e. between the side with the ionic components and the side with neutral precursors. The formation of the ionic liquid is driven by the enthalpy of the neutralization reaction and dominated by Coulomb forces and additional hydrogen bonds. This reaction enthalpy gets more exothermic with increasing basicity of the base and increasing acidity of the acid. The farther the equilibrium is on the side of the ionic liquid, the lower the volatility of the ionic liquid is and the higher the pseudo boiling point of the ionic liquid is.

The relation between these factors was previously described for protic ionic liquids (Yoshizawa, M., Xu, W., Angell, C. A.; J. Am. Chem. Soc. 125, 15411-15419 (2003). DOI: 10.1021/ja035783d). Yoshizawa et al. defined an “excess boiling point” ATb, as the difference between i) an experimentally measured boiling of the ionic liquid, i.e. the (pseudo) boiling point T_{b}, and ii) the arithmetic mean of the two boiling points of the precursors (T_{b} B and T_{b} HA), i.e. the base B and the acid HA, which react to the particular ionic liquid. They found that a strong linear correlation between ΔT_{b} and Brønsted activity for some distillable acid-base conjugate ionic liquids. The Brønsted activity being expressed as ΔpKₐ, which is the difference of the two aqueous pKₐ values of the precursors, i.e. the pKₐ of the corresponding acid conjugated to the base B, minus the pKₐ the of the acid HA. For a number of seven diverse, distillable acid-base conjugate ionic liquids, they found a strong linear correlation between ΔT_{b} and ΔpKₐ. The inventors have extended this analysis to 30 ionic liquids (see Table 1 and FIG. 6). The high linear correlation between ΔT_{b} and ΔpKₐ was confirmed with a Pearson correlation coefficient of R²=0.9696.

TABLE 1
Pseudo boiling point of acid-base conjugate ionic liquids (T_{b} IL) in comparison to boiling points and
pKₐ values of their precursor acids HA (T_{b} HA, pKₐ HA, ac. refers to acid) and precursor bases B (T_{b} B, pKₐ B),
arithmetic means of the boiling points of the precursors (T_{b} HA + B), excess boiling temperature (ΔT_{b}, i.e. T_{b} IL
minus T_{b} HA + B) and difference between the pKₐ values of the precursor (ΔpKₐ).

| | | T_{b} | | | T_{b} | | T_{b} | T_{b} | | | |
|---|---|---|---|---|---|---|---|---|---|---|---|
| | Name | HA | pKₐ | Name | B | pKₐ | HA + B | IL | ΔT_{b} | | |
| ID | acid HA | ° C. | HA | base B | ° C. | B | ° C. | ° C. | ° C. | ΔpKₐ | Source |
| 1 | N,N-Dimethyl- | −23^{l} | 6.31^{k} | Dimethylamine | 7 | 10.71 | −8 | 61 | 69 | 4.4 | a, b |
| | carbamic ac. | | | | | | | | | | |
| 2 | CO₂ | −78^{m} | 6.35 | Diethylamine | 56 | 10.98 | −11 | 62 | 73 | 4.63 | b, c |
| 3 | N,N-diethyl- | −23^{l} | 6.31^{k} | Diethylamine | 56 | 10.98 | 16.5 | 63 | 46.5 | 4.67 | a, b |
| | carbamic ac. | | | | | | | | | | |
| 4 | CO₂ | −78^{m} | 6.35 | Pyrrolidine | 87 | 11.27 | 4.5 | 80 | 75.5 | 4.92 | b, c |
| 5 | Formic ac. | 101 | 3.75 | Pyridine | 115 | 5.23 | 108 | 113 | 5 | 1.48 | d |
| 6 | Formic ac. | 101 | 3.75 | N-Methyl- | 115 | 7.38 | 108 | 136 | 28 | 3.63 | e |
| | | | | morpholine | | | | | | | |
| 7 | Formic ac. | 101 | 3.75 | N-Ethyl- | 138 | 7.70 | 119.5 | 137 | 17.5 | 3.95 | e |
| | | | | morpholine | | | | | | | |
| 8 | Formic ac. | 101 | 3.75 | α-Picoline | 128 | 5.96 | 114.5 | 142 | 27.5 | 2.21 | f |
| 9 | Formic ac. | 101 | 3.75 | Dimethylamine | 7 | 10.71 | 54 | 152 | 98 | 6.96 | c, g |
| 10 | Formic ac. | 101 | 3.75 | Morpholine | 129 | 8.36 | 115 | 152 | 37 | 4.61 | e |
| 11 | Formic ac. | 101 | 3.75 | Triethylamine | 89 | 10.76 | 95 | 153 | 58 | 7.01 | h |
| 12 | Acetic ac. | 118 | 4.76 | Ethylamine | 17 | 10.75 | 67.5 | 156 | 88.5 | 5.99 | c, g |
| 13 | Formic ac. | 101 | 3.75 | Methylamine | −6 | 10.66 | 47.5 | 162 | 114.5 | 6.91 | c, g |
| 14 | Formic ac. | 101 | 3.75 | Ethylamine | 17 | 10.75 | 59 | 162 | 103 | 7 | c, g |
| 15 | Formic ac. | 101 | 3.75 | n-Propylamine | 47 | 10.57 | 74 | 165 | 91 | 6.82 | f |
| 16 | Formic ac. | 101 | 3.75 | Ethanolamine | 172 | 9.5 | 136.5 | 192 | 55.5 | 5.75 | c, g |
| 17 | Butyric ac. | 164 | 4.82 | Ethanolamine | 172 | 9.5 | 168 | 198 | 30 | 4.68 | i |
| 18 | Trifluoroacetic | 72.5 | 0.50 | α-Picoline | 128 | 5.96 | 100.25 | 200 | 99.75 | 5.46 | f |
| | ac. | | | | | | | | | | |
| 19 | Trifluoroacetic | 72.5 | 0.50 | n-Propylamine | 47 | 10.57 | 59.75 | 207 | 147 | 10.07 | f |
| | ac. | | | | | | | | | | |
| 20 | Nitric ac. | 86 | −1.37 | Methylamine | −6 | 10.66 | 40 | 207 | 167 | 12.03 | c, g |
| 21 | Acetic ac. | 118 | 4.76 | Ethanolamine | 172 | 9.5 | 145 | 210 | 65 | 4.74 | c, g |
| 22 | Glycolic ac. | 100 | 3.83 | Ethanolamine | 172 | 9.5 | 136 | 216 | 80 | 5.67 | c, g |
| 23 | Phosphoric ac. | 213 | 2.16 | Ethylamine | 17 | 10.75 | 115 | 252 | 137 | 8.59 | c, g |
| 24 | Methanesulfonic | 217 | −1.9 | Dimethylamine | 7 | 10.71 | 112 | 278 | 166 | 12.61 | c, g, j |
| | ac. | | | | | | | | | | |
| 25 | Nitric ac. | 86 | −1.37 | Ethanolamine | 172 | 9.5 | 129 | 280 | 151 | 10.87 | c, g |
| 26 | Sulphuric ac. | 290 | −3 | Dimethylamine | 7 | 10.71 | 148.5 | 311 | 162.5 | 13.71 | c, g |
| 27 | Methanesulfonic | 217 | −1.9 | Triethylamine | 89 | 10.76 | 153 | 329 | 176 | 12.66 | h, j |
| | ac. | | | | | | | | | | |
| 28 | Triflic ac. | 162 | −14 | α-Picoline | 128 | 5.96 | 145 | 450 | 305 | 19.96 | f |
| 29 | Hydrochloric ac. | −85 | −6.2 | Pyridine | 115 | 5.23 | 15 | 222 | 207 | 11.43 | a |
| 30 | Formic ac. | 101 | 3.75 | N-Methyl- | 198 | 6.95 | 150 | 174 | 25 | 3.2 | a, h |
| | | | | imidazole | | | | | | | |

Sources: a CAS SciFinder™ (SciFinder; Chemical Abstracts Service: Columbus, OH; https://scifinder.cas.org; values accessed January 2022); b Schroth, W., Schädler, H.-D., Andersch, J. Z. Chem. 29 (4) (1989) 129-135; c Wikipedia DE (Wikipedia; https://de.wikipedia.org; German language or English language article on respective compound accessed January 2022); d Rashid, T., Kait, C. F., Murugesan, T. Chinese Journal of Chemical Engineering 25 (2017) 1266-1272; e Brigouleix, C., Anouti, M., Jacquemin, J. et al. J Phys Chem B 114 (2010) 1757-1766; f Yoshizawa, M., Xu, W., Angell, C. A.; J. Am. Chem. Soc. 125, 15411-15419 (2003). DOI: 10.1021/ja035783d); g Greaves, T. L., Drummond, C. J., Chem Rev 108 (2008) 206-237; h Proionic GmbH (in house data by TGA); i Pinto, R. R., Mattedi, S., Aznar, M. Chemical Engineering Transactions 43 (2015) 1165-1170; j Avantor® delivered by VWR® (https://at.vwr.com/store/product/2358460/methansulfonsaure-sigma-aldrich accessed February 2022); k predicted pKₐ by CAS SciFinder™ (SciFinder; Chemical Abstracts Service: Columbus, OH; https://scifinder.cas.org; values accessed January 2022); 1 thermal decomposition point of the analogue carbamic acid is −23° C. (R. K. Khanna and M. H. Moore, Spectrochimica Acta Part A 55 (1999) 961-967, DOI: 10.1016/S1386-1425 (98) 00228-5); m sublimation point at 1 atm, Wikipedia DE (Wikipedia; https://de.wikipedia.org, accessed February 2022).

Based on the correlation between ΔT_{b} and ΔpKₐ, the following preferences for the selection of the neutral nitrogen compound (base B) and the acid (HA), forming the distillable acid base conjugate salt ionic liquid BH⁺A⁻ were identified:

A lower ΔpKₐ is accompanied by a lower excess boiling point ΔT_{b} for the ionic liquid (see FIG. 6) and thus, allows for precursor compounds that have higher boiling points (i.e., higher arithmetic mean of T_{b} HA and T_{b} B). For example, if the ΔpKₐ is about 10, ΔT_{b} is about 145° C. and thus, to meet the preferred maximum pseudo boiling point of about 210° C., the precursors must have a boiling point T_{b} HA+B as low as 55° C. (calculated arithmetic mean of T_{b} HA and T_{b} B). If, however, ΔpKₐ is about 5, ΔT_{b} is about 70° C. and thus, to meet the preferred maximum pseudo boiling point of about 210° C., the precursor can have a boiling point T_{b} HA+B up to 130° C. (calculated arithmetic mean of T_{b} HA and T_{b} B).

Accordingly, in a preferred embodiment, the acid base conjugate salt ionic liquid has ΔpKₐ of ≤10, preferably ≤7.5, such as ≤6.0 or ≤5.0, wherein the ΔpKₐ is the difference between the aqueous pKₐ value of the corresponding acid conjugated to the base B and the aqueous pKₐ of acid HA, from which base B and acid HA the ionic liquid is derived.

In a further embodiment, it is preferred that the boiling point of the base B (T_{b} B) and the boiling point of the acid HA (T_{b} HA), from which base B and acid HA the ionic liquid is derived, are similar at a given pressure, in particular T_{b} B≥T_{b} HA.

After the pseudo boiling point of the ionic liquid, the relation of the boiling points of the precursor compounds to each other is a second line criteria for selection of preferred ionic liquids. In cases, wherein the boiling point of the base B and the boiling point of the acid HA, of which an ionic liquid is formed, differ much from each other, this can lead to an enrichment of the higher boiling compound during the pseudo boiling of the ionic liquid. In such a case of asymmetric evaporation, two steps of mass loss can be detected in the TGA and two peaks could appear in the DSC experiment. An ideal ionic liquid would neither enrich B nor HA during transformation in gaseous form, which is the case if the two boiling points T_{b} HA and T_{b} B are close to each other as well as the two enthalpies of evaporation.

However, an enrichment of base B (being a neutral nitrogen compound and potentially suited as medium as well) may be a neglectable disadvantage, compared to an enrichment of the acid HA (potentially being corrosive or aggressively reacting with the biomass in an undesired manner). Therefore, the boiling point of the acid HA is preferably close to equal or less than the boiling point of the base B at a given pressure, i.e., T_{b} B≥T_{b} HA.

In a preferred embodiment, the acid, from which the anion of an acid base conjugate salt ionic liquid is derived, is an acid selected from the group of
carboxylic acids R—COOH, wherein R is selected from alkyl groups with 1 to 6 carbon atoms, alkenyl groups with 2 to 6 carbon atoms, and aryl groups with 6 carbon atoms, wherein any alkyl, alkenyl or aryl group is unsubstituted or substituted with a hydroxyl group,
phenols (hydroxybenzenes), wherein the phenyl of the phenol is unsubstituted or substituted with a methoxy or methyl group, such as phenol, 2-methoxyphenol, o-, m- and p-cresol, and
carbonic acid or carbon dioxide (CO₂).

Any alkenyl group with 2 to x carbon atoms is also referred to as C₂-Cₓ-alkenyl includes at least one double bond, such as one or two, preferably one, double bond and is to be understood to include linear C₂-Cₓ-alkenyl (such as vinyl, propenyl, allyl, etc.), branched C₃-Cₓ-alkenyl (such as isoprenyl) and cyclic C₃-Cₓ-alkenyl (such as cyclopentenyl).

Preferred acids are carboxylic acids with no halogen atoms, especially no fluorine atoms.

More preferably, the acid is selected from the group consisting of formic acid, acetic acid, propionic acid, butyric acid, valeric acid, caproic acid, acrylic acid, glycolic acid, lactic acid, phenol, and salicylic acid.

These preferred acids are selected based on their commercial availability as well as their boiling point being at about 210° C. or below at normal pressure.

In a preferred embodiment, the boiling point of the acid (T_{b} HA) forming the anion of the ionic liquid is about 200° C. or below, more preferably 175° C. or below, such as below 150° C.

Additionally, the neutral nitrogen compound forming the cation is preferably a compound according to formula B, wherein none of R₁, R₂ and R₃ is hydrogen, i.e., the compound is—in the widest sense—a tertiary amine. Such neutral nitrogen compounds have the advantage that they do not form an acid amide upon contact with an acid, whereas primary or secondary amines may undergo an undesired side-reaction with the acid and form an acid amide, e.g., a carboxamide, by condensation at the conditions of the method according to the invention (i.e., reduced pressure, elevated temperature).

Accordingly, the neutral nitrogen compound forming the cation is preferably selected from the group consisting of trimethylamine, triethylamine, tripropylamine, tributylamine, dimethylethylamine, dimethylpropylamine, dimethylbutylamine, N,N-dimethylethanolamine, pyridine, N-methylmorpholine, N-ethylmorpholine, N-methylpiperidine, N-ethylpiperidine, N-methylimidazole, N-ethylimidazole, N-methylpyrrolidine, N-ethylpyrrolidine, N-butylpyrrolidine, and N,N,N′,N′-tetramethylethylendiamine.

In a preferred embodiment, the acid base conjugate ionic liquid is selected from the group of butylammonium formate, dibutylammonium formate, diethylammonium formate, dimethylammonium formate, dipropylammonium formate, ethanolammonium formate, ethylammonium formate, methylammonium formate, morpholinium formate, N-ethylimidazolium formate, N-ethylmorpholinium formate, N-methylimidazolium formate, N-methylmorpholinium formate, N-methylpyrrolidinium formate, propylammonium formate, pyridinium formate, pyrrolidinium formate, tributylammonium formate, triethylammonium formate, trimethylammonium formate, tripropylammonium formate, α-picolinium formate, butylammonium acetate, dibutylammonium acetate, diethylammonium acetate, dimethylammonium acetate, dipropylammonium acetate, ethanolammonium acetate, ethylammonium acetate, methylammonium acetate, morpholinium acetate, N-ethylimidazolium acetate, N-ethylmorpholinium acetate, N-methylimidazolium acetate, N-methylmorpholinium acetate, N-methylpyrrolidinium acetate, propylammonium acetate, pyridinium acetate, pyrrolidinium acetate, tributylammonium acetate, triethylammonium acetate, trimethylammonium acetate, tripropylammonium acetate, α-picolinium acetate, butylammonium propionate, dibutylammonium propionate, diethylammonium propionate, dimethylammonium propionate, dipropylammonium propionate, ethanolammonium propionate, ethylammonium propionate, methylammonium propionate, morpholinium propionate, N-ethylimidazolium propionate, N-ethylmorpholinium propionate, N-methylimidazolium propionate, N-methylmorpholinium propionate, N-methylpyrrolidinium propionate, propylammonium propionate, pyridinium propionate, pyrrolidinium propionate, tributylammonium propionate, triethylammonium propionate, trimethylammonium propionate, tripropylammonium propionate, α-picolinium propionate, butylammonium butyrate, dibutylammonium butyrate, diethylammonium butyrate, dimethylammonium butyrate, dipropylammonium butyrate, ethanolammonium butyrate, ethylammonium butyrate, methylammonium butyrate, morpholinium butyrate, N-ethylimidazolium butyrate, N-ethylmorpholinium butyrate, N-methylimidazolium butyrate, N-methylmorpholinium butyrate, N-methylpyrrolidinium butyrate, propylammonium butyrate, pyridinium butyrate, pyrrolidinium butyrate, tributylammonium butyrate, triethylammonium butyrate, trimethylammonium butyrate, tripropylammonium butyrate, α-picolinium butyrate, butylammonium lactate, dibutylammonium lactate, diethylammonium lactate, dimethylammonium lactate, dipropylammonium lactate, ethanolammonium lactate, ethylammonium lactate, methylammonium lactate, morpholinium lactate, N-ethylimidazolium lactate, N-ethylmorpholinium lactate, N-methylimidazolium lactate, N-methylmorpholinium lactate, N-methylpyrrolidinium lactate, propylammonium lactate, pyridinium lactate, pyrrolidinium lactate, tributylammonium lactate, triethylammonium lactate, trimethylammonium lactate, tripropylammonium lactate, and α-picolinium lactate.

Furthermore, the definition of the nitrogen compound also includes the class of alkyl carbamate ionic liquids (a subclass of nitrogen-based protic ionic liquid), which formalistically derive from a secondary amine or—less preferable—a primary amine and CO₂. They are also referred to as (distillable) CO₂-based alkyl carbamate ionic liquids. The formation of an alkyl carbamate ionic liquid may be described according to the equation III:

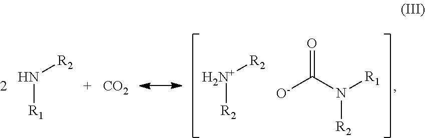

wherein R₁ and R₂ independently from each other are selected from hydrogen and alkyl groups with 1 to 6 carbon atoms, preferably both R₁ and R₂ are selected from alkyl groups with 1 to 6 carbon atoms,
or R₁ and R₂ together with the nitrogen atom form 5-6 membered heterocyclic group,
wherein any alkyl group independently from each other may be unsubstituted or substituted with a hydroxyl group or a further NR₃R₄ group, wherein R₃ and R₄ independently from each other are selected from hydrogen and unsubstituted alkyl groups with 1 to 3 carbon atoms,
preferably both R₃ and R₄ are selected from unsubstituted alkyl groups with 1 to 3 carbon atoms, and
wherein any heterocyclic group may be aliphatic or aromatic and may further comprise one to two further heteroatoms selected from O, S, N, and NR₅, wherein R₅ is selected from hydrogen or unsubstituted alkyl groups with 1 to 4 carbon atoms, preferably R₅ is selected from unsubstituted alkyl groups with 1 to 4 carbon atoms.

Thus, the secondary amine involved in forming an alkyl carbamate ionic liquid (R₁R₂NH) may be defined as a compound according to formula B, wherein at least R₃ is hydrogen.

As the acid base conjugate ionic liquid, an alkyl carbamate ionic liquid may be referred to as distillable. The alkyl carbamate ionic liquid is in equilibrium with neutral compounds, from which is it derived. The considerations and preferences, discussed for the pseudo boiling point and the selection of the preferred base of the distillable acid base conjugate ionic liquid analogously apply for the alkyl carbamate ionic liquid.

In a preferred embodiment, the alkyl carbamate ionic liquid is selected from the group of carbamate ionic liquids, wherein R¹ and R₂ independently from each other are selected from ethyl, methyl, propyl, pyrrolidinium pyrrolidincarbamate (CAS 1349851-87-1), piperidinium piperidincarbamate (CAS 2455-83-6) and morpholinium morpholincarbamate (CAS 62038-13-5).

In a further aspect, the nitrogen compound may be a hybrid form of neutral nitrogen compounds and ionic liquids. The term hybrid form is understood to include mixtures of a neutral nitrogen compound and an ionic liquid in any quantitative relation to each other. In one embodiment, the hybrid form comprises a neutral nitrogen compound and an ionic liquid, wherein the cation of the ionic liquid is derived from said neutral nitrogen compound. The term hybrid form also covers a nitrogen compound representing a mixture of an ionic liquid with a protonated nitrogen compound as cation with a different (i.e., unrelated) neutral nitrogen compound. Preferably, the hybrid form comprises a neutral nitrogen compound according to formula B and an ionic liquid according to formula C, wherein the cation BH_{y}^{y+}, preferably BH⁺, is a protonated form of the neutral nitrogen compound according to formula B. Such hybrid forms can be obtained by mixing a compound of formula B with a sub-stochiometric amount of an acid.

As discussed above, neutral nitrogen compounds and nitrogen-based protic ionic liquids, preferably ionic liquids being selected from acid base conjugate salts and alkyl carbamate ionic liquids, and hybrid forms thereof, all include compounds that readily vaporize under the pressure and temperature conditions preferred for the method according to the invention, e.g. at a pressure of from 10⁻¹ to 100 mbar, such as 1 mbar or 10 mbar, and at a temperature of from 80° C. to 140° C., such as 100° C. Thus, they are especially suited for the method according to the present invention.

In a preferred embodiment, the medium has a content of the nitrogen compound in the range of from 10 to 100 wt. %, preferably from 50 to 100 wt. %, more preferably from 90 to 100 wt. % such as essentially 100 wt. %.

Whereas it may be preferred that the medium essentially consists of the nitrogen compound as defined above, in further embodiments, the medium comprises one or more further component(s). In an embodiment, further component(s) of the medium are selected from the group consisting of co-solvents, buffers, detergents, dispergents, anticorrosants, and antioxidants.

Suitable co-solvents include for example water, alcohols (such as methanol, ethanol, 1-propanol, 2-propanol, 1-butanol, 2-butanol, and ethylenglycole), ethers (such as ethylenglycoldimethylether, ethylenglycoldiethylether, ethylenglycolmonoethylether, ethylenglycolmonobutylether, dipropylether, di-n-butylether, di-iso-butylether, di-sec-butylether, di-tert-butylether, 2-methoxy-2-methylpropan), dihydrolevoglucosenon (Cyrene™), isosorbid-dimethylether, levoglucosenone, γ-valerolactone, carbonic acids (such as formic acid, acetic acid, propionic acid, and lactic acid), DMSO, 2,5-dimethylfuran, triethylphosphate, acetone and dissolved CO₂.

In one embodiment, the biomass material remaining collectable after the removal of the nitrogen compound is obtained in combination with a co-solvent, in particular the co-solvent from the medium. In this embodiment, the biomass is not retrieved as dry solid but kept in a moist or wet state by the co-solvent such as water or alcohol. This is desirable in situations, wherein the remaining biomass material should not dry out, for example a biomass material comprising intact cellular structures. Preferably, the remaining biomass material may be collected in combination with a co-solvent, in particular water, wherein the co-solvent, in particular water, is present in a concentration of from 5 to 30 wt. %, preferably 10 to 25 wt. %, such as 20 wt. % with respect to said combination. Typically, the co-solvent, such as water or alcohol, forms a gaseous form, evaporates and is removed with the gaseous phase in the method according to the invention. However, supplementation of co-solvent during the processing allows to maintain a certain concentration of co-solvent, while the nitrogen compound is removed. Accordingly, in this embodiment, the method can further comprise adding a co-solvent, in particular continuously adding a co-solvent, preferably water, while removing the gaseous phase.

A neutral nitrogen compound as defined above may act as a co-solvent. Indeed, a neutral nitrogen compound may be advantageously added to an ionic liquid, which results in a hybrid form of the neutral nitrogen compound and the ionic liquid as described above.

Suitable buffers include for example ammonium carbonate, ammonium hydrogencarbonate, ammonium carbamate. Of note, neutral nitrogen compounds or ionic liquids themselves can act as buffer systems.

In a preferred embodiment, any one of further component(s) is distillable or thermosensitive. Thus, each of the further component(s) may transformed into a gaseous form and removed from the mixture during the method according to the invention. More preferably, any one of further component(s) is distillable under the conditions of the method according to the invention (temperature, pressure) and preferably recovered with nitrogen compound.

In one embodiment, the relation between the biomass material comprised in the initial mixture as initially provided and the remaining biomass is that the first is a raw material for the later. As the nitrogen compound (or the medium) can be removed efficiently, the remaining biomass material is substantially free of the nitrogen compound (or free of the medium).

In a preferred embodiment, the biomass material is a lignocellulosic biomass and/or a cellulosic biomass material. For example, a biomass material originating from
agricultural residues of any kind, such as husk, straw, stover, spelts, seeds, leaves, empty fruit bunches and the like, exemplary oil palm empty fruit bunch (i.e. a residual biomass remaining from Elaeis guineensis from collecting the oil-rich seeds),
forestry and orchard waste, or
energy crops, such as switchgrass, Sorghum bicolor, poplar, hemp, and giant miscanthus.

In general, the biomass material can be any woody biomass, grassy biomass, herbaceous biomass etc., containing polysaccharides such as glucan or xylan.

In another preferred embodiment, the biomass material is or contains proteins-such as wool, feathers, in particular the proteins silk, keratin, collagen, gorgonin (or a protein hydrolysate derived from a protein), or polyaminosaccharides, such as chitin, chitosan, heparin, chondroitin, dermatan, keratan and hyaluronic acid.

Furthermore, the nitrogen compound (or the medium) may be recovered with high purity. Preferably, the nitrogen compound is recovered with a recovery rate of 98 wt. % or above, preferably 99 wt. % or above, most preferably 99.5 wt. % or above with respect to the amount of nitrogen compound in the initial mixture. If necessary, the recovered medium may be subjected to further cleaning steps. For example, volatile compounds of the biomass material which are removed together with the gaseous stream or undesired components of the medium can be removed from nitrogen compounds being ionic liquids by mild distillation conditions.

The method of the invention can be usefully integrated in processes to produce a sugar or to produce biofuel (such as ethanol, butanol or hydrocarbons) from a biomass as discussed in Example 4. Thus, in a further aspect, the present invention relates to a process for producing a sugar or biofuel from a biomass.

Exemplarily, a process for producing a sugar or biofuel from a biomass comprises the following steps of
(a) generating a mixture comprising the biomass material and a pretreatment medium comprising a nitrogen compound and pretreating the mixture under condition that allow to at least partially solubilize the biomass material and/or disintegration and/or transformation of the (cellular) structure of the biomass material,
(b) separating the pretreatment medium comprising a nitrogen compound by applying the method according to the invention to obtain a remaining biomass, and preferably, recovering the pretreatment medium comprising a nitrogen compound,
(c) transferring the remaining biomass material into an essentially aqueous medium,
(d) hydrolyzing the biomass, comprising introducing an enzyme and/or a microbe, such that the enzyme and/or microbe produce a sugar from the biomass, resulting in a hydrolysate
(e) optionally fermenting the sugar in the hydrolysate to a biofuel such as ethanol, butanol or hydrocarbons, resulting in a fermentation mixture,
(f) optionally separating the biofuel from the fermentation mixture by distillation,
(g) optionally separating unprocessed (non-hydrolysable) biomass material from the fermentation mixture by solid liquid separation, and,
(h) optionally performing the process in a recycling setup and reusing the recovered pretreatment medium comprising a nitrogen compound in step (a).

In one embodiment, the step (a) of pretreating the biomass conveniently can be performed in the same apparatus as the method according to the invention. Indeed, both thin-film processors as well as large-volume processors as discussed above may be used for the pretreatment step as also during pretreatment, high sheering forces and intensive mixing may be preferred.

In the following, non-limiting figures and examples are discussed to visualize and demonstrate certain aspects of the invention.

### BRIEF DESCRIPTION OF THE DRAWINGS

FIG. 1 is a graph showing the recovery of the ionic liquid ethanolammonium acetate over time from a pretreated mixture additionally comprising a biomass material originating from Sorghum bicolor from a static film of the biomass with a thickness of about 1 mm (squares) in comparison to recovery with a conventional rotary evaporator (triangles) at a temperature of 140° C. and a pressure of 1 mbar.

FIG. 2 is graph showing the recovery of the neutral nitrogen compound butylamine over time from a pretreated mixture additionally comprising the biomass material spelt husk by using a conventional rotary evaporator (triangles) and a laboratory model of a large-volume processor (squares) at a temperature of 80° C. and a pressure of 40 mbar.

FIG. 3A is graph showing the recovery of the ionic liquid triethylammonium formate over time from a pretreated mixture additionally comprising the biomass material spelt husk by using a conventional rotary evaporator (triangles) and a laboratory model of a large-volume processor (squares) at a temperature of 120° C. and a pressure of 40 mbar.

FIG. 3 B is graph showing the recovery of the ionic liquid pyridinium formate over time from a pretreated mixture additionally comprising the biomass material spruce sawdust by using a conventional rotary evaporator (triangles) and a laboratory model of a large-volume processor (squares) at a temperature of 100° C. and a pressure of 100 mbar.

FIG. 4 is a schematic depiction of a thin-film processor.

FIG. 5 is a schematic depiction of a large-volume processor.

FIG. 6 is a graph showing the relation of the excess boiling point (ΔT_{b}) with the difference in aqueous solution pKₐ values for the base B and the acid HA (ΔpKₐ) forming the respective acid base conjugate ionic liquids, wherein the ΔT_{b} is determined as the difference between the pseudo boiling point and the arithmetic mean of the boiling point of the pure acid and the pure base.

FIG. 7 is a schematic depiction of a set-up to study used to investigate a static evaporation surface from top (left depiction) and side (right depiction).

FIG. 8 is a chart of a process for producing biofuel (ethanol) from a mixture comprising a biomass material and a nitrogen compound, e.g., an ionic liquid, according to the state of the art.

FIG. 9 is a chart of a process for producing biofuel (ethanol) from a mixture comprising a biomass material and a nitrogen compound, e.g., an ionic liquid, using a method according to the invention for separating and recovering the nitrogen compound.

### EXAMPLES

### Example 1: Evaluation of Recovery of a Pretreatment Medium Comprising an Ionic Liquid from a Biomass Mixture with a Static an Evaporation Surface

This preliminary experiment aimed at studying the evaporation of an ionic liquid (IL) from a biomass mixture through a static evaporation surface.

A mixture comprising a biomass material was prepared by the following pretreatment process: The grass Sorghum bicolor was dried for 24 h at 40° C. and subsequently knife-milled to a grain size of 2 mm. 1.5 kg of the bone-dry material was transferred together with 8.5 kg of the distillable IL ethanolammonium acetate (CAS 54300-24-2, T_{b}=210° C.) into a 20 L pressure tight stainless steel autoclave (Kiloclave, Buechi AG) equipped with an impeller stirrer, and heated for 3 hours at 140° C. under stirring at 100 rpm. After this pretreatment, the mixture comprising biomass and IL showed a pasty consistence and a dark brown color.

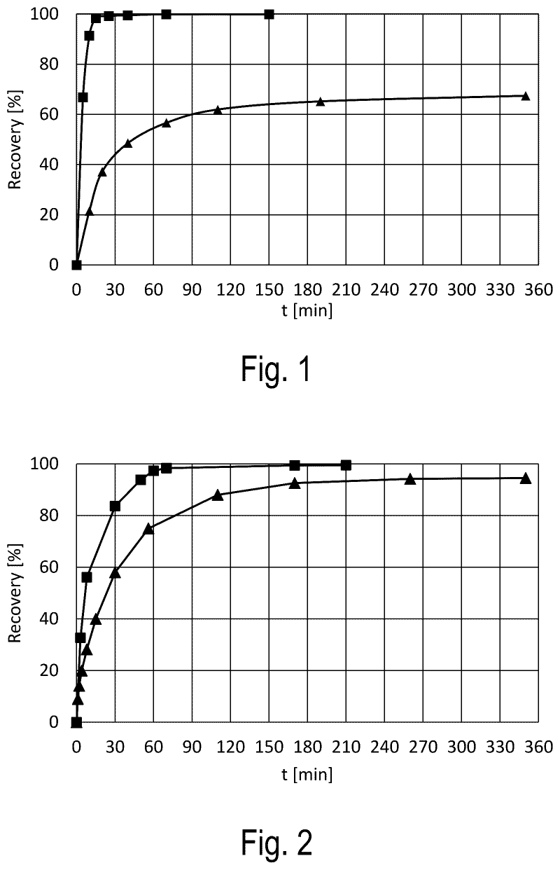

In a first experiment, 4 g of this pretreated biomass mixture was weighed into a 50 mL round bottom flask with a precision of 1 mg. The sample was then adjusted to a standard rotary evaporator and rotated at 50 rpm; then the ionic liquid was distilled at a constant vacuum of 1 mbar and at 140° C. The initially formed film had a thickness of approximately 5 mm; in the course of the ionic liquid removal the biomass turned into a very sticky, dark brown and hard mass. The recovery of the distillate was measured as mass loss and plotted as a function of time, until no change in mass was observable anymore (FIG. 1, triangles). Blank experiments of untreated biomass, containing no ionic liquid, resulted in neglectable mass losses.

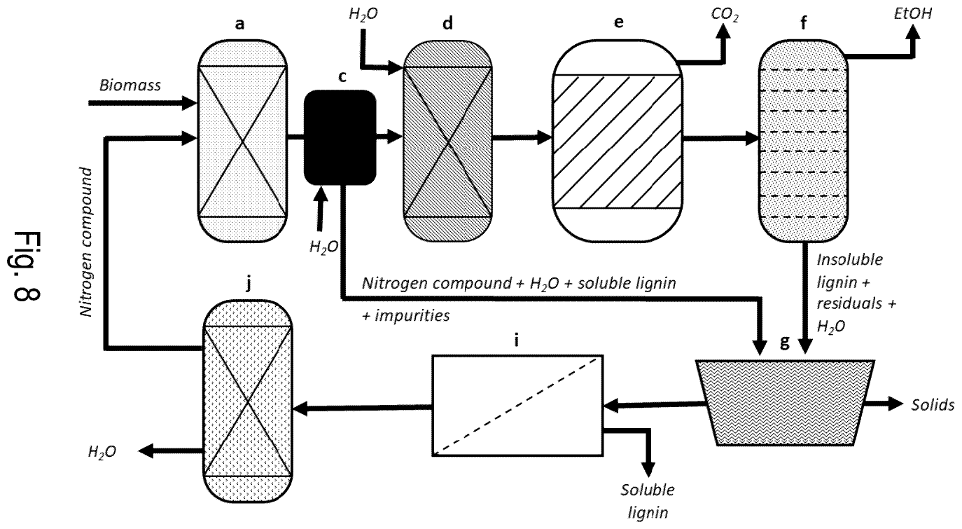

In a second experiment, the evaporation of the ionic liquid through a static but thin evaporation surface was studied. The setup imitates a single evaporation surface in a thin-film processor under stopped rotation of the central axis. For this a wiped-film short path distillation device with 4 dm² internal surface and a central, coaxial condenser (Manufacturer VTA Verfahrenstechnische Anlagen GmbH & Co. KG, Niederwinkling, Germany) was modified as depicted in FIG. 7.

FIG. 7 shows two schematic sectional views of the evacuable vessel 1 of the device, a sectional view from top on the left side and a sectional side view on the right side.

The distribution unit containing the wiper was removed from the device. A thin, curved magnetic steel plate 5 (with approx. 0.75 dm² and a radius fitting exactly to the internal surface of the device and fixed weight determined with an accuracy of 1 mg) was mounted (i.e. removably fixed) in the vessel 1 using a strong magnet 6. The steel plate 5 had a U-shaped lower edge, to prevent losing biomass particles eventually falling down during the process from the steel plate and subsequently not being weighed (see side view).

Subsequently, 4 g of the biomass-IL mixture 4 were distributed on the metal plate 5, to form a static film with a thickness of about 1 mm. The mass of the biomass film was weight with an accuracy of 1 mg.

The film of the mixture 4 thus was exposed to a temperature of 140° C. and a pressure of 1 mbar, the condenser 7 was cooled with tap water. Colorless, clear droplets of the distilled ionic liquid were formed on the condenser, which were collected after the experiment and compared to fresh ionic liquid by means of 1H-NMR, showing identical signals.

The weight loss of on the metal plate was determined every 5 min to evaluate the evaporation from the static layer and thus, the recovery of the IL (FIG. 1, squares).

Thus, the preliminary results suggested that methods, which could ensure the provision of a large evaporation surface, are useful for allowing the distillable ionic liquid to evaporate in the gaseous form, remove it from the vessel and further recover it by condensing. The recovery rate was determined to be 99.9 wt. %. In contrast to this, when the biomass mixture formed incrustions and thicker films, the ionic liquid's evaporation was slowed and overall limited to a recovery rate below 70%.

### Example 2A: Evaluation of the Mechanical Processing on Recovery of a Pretreatment Medium Comprising a Neutral Nitrogen Compound or an Ionic Liquid

A biomass material containing husk spelts was provided in the form of mixtures with two different (pre) treatment media comprising either the neutral nitrogen compound n-butylamine or the ionic liquid triethylammonium formate and both processed in different apparatuses to study the effect of the mechanical processing during the method according to the invention.

The first biomass mixture was prepared by the following pretreatment process: The husk spelts were dried for 24 h at 40° C. and subsequently knife-milled to a grain size of 2 mm. 1.5 kg of the bone-dry material was transferred together with 8.5 kg of the nitrogen compound n-butylamine (CAS 109-73-9, T_{b}=78° C. @ 1 atm) into a 20 L pressure tight stainless steel autoclave (Kiloclave, Buechi AG) equipped with an impeller stirrer, and heated for 2 hours at 120° C. under stirring at 100 rpm. After this treatment the mixture showed a pasty consistence and a dark brown color.

The second biomass mixture was prepared by the following pretreatment process: The husk spelts were dried for 24 h at 40° C. and subsequently knife-milled to a grain size of 2 mm. 750 g of the bone-dry material was transferred together with 4250 g of triethylammonium formate (CAS 585-29-5, T_{b}=153° C. @ 1 atm, ID 11 in Table 1) into a 20 L pressure tight stainless steel autoclave (Kiloclave, Buechi AG) equipped with an impeller stirrer and heated for 3 hours at 100° C. under stirring at 100 rpm. After this treatment the mixture showed a pasty consistence and a dark brown color.

The mixtures were independently feed into a) conventional rotary evaporator (Heidolph Instruments GmbH & CO. KG, Schwabach, Germany) and b) a laboratory model of a large-volume processor (Werner & Pfleiderer Lebensmitteltechnik GesmbH, formerly Werner & Pfleiderer A G, Vienna, Austria). The large-volume processor model was equipped with two rotating, Z-shaped kneading elements, wherein these elements are arranged to rotate within the vessel, such that the complete load of the vessel is circulated. The two elements almost touch themselves throughout a full rotation, with a clearance of approx. 0.5 mm or less, and are therefore “self-cleaning”.

Both apparatuses provide an evacuable vessel, wherein the internal surface can be heated with an inner volume of 2 liter. Both apparatuses were operated with a rotational speed of 50 rpm, which results in a peripheral speed of about 0.22 m/s for the lab-scale large-volume processor. A pressure of 40 mbar was applied in all experiments and the vessels were heated to a temperature of 80° C. and 120° C. for the nitrogen compound and the ionic liquid, respectively.

In the conventional rotary evaporator, recovery of the pretreatment medium was determined as a function of time by the mass loss of the mixture. In the lab-scale large-volume processor, the actual recovery of the pretreatment media was determined as it was condensed downstream using a cold trap operated with liquid nitrogen.

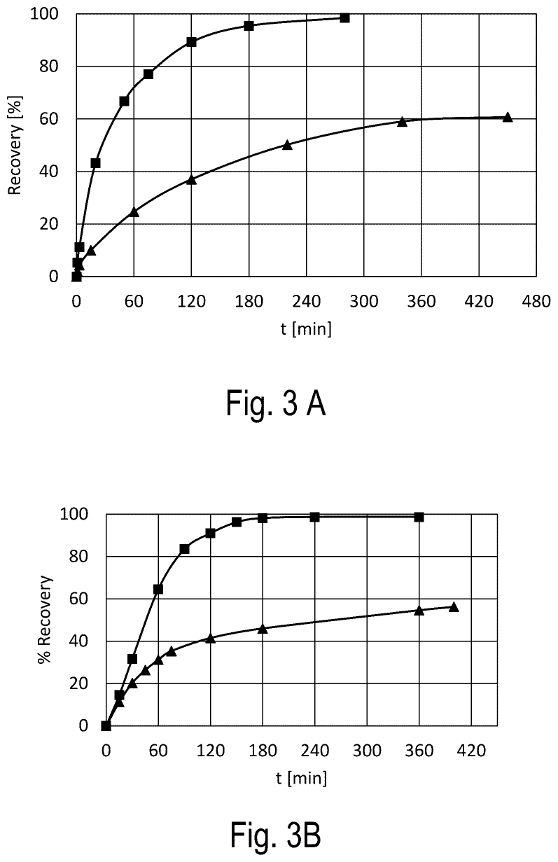

The results for the pretreatment medium comprising the neutral nitrogen compound n-butylamine and the ionic liquid triethylammonium formate are shown in FIGS. 2 and 3A, respectively, for both conventional rotary evaporator (triangles) and a laboratory model of a large-volume processor (squares) and final recovery rates summarized in Table 2 below.

TABLE 2
Recovery of the nitrogen compound using different
processing apparatuses and pretreatment media

| | Processing apparatus | | | |
|---|---|---|---|---|
| | lab scale large-volume processor | | conventional rotary evaporator | |
| Nitrogen | max. recovery | | max. recovery | |
| compound as | of nitrogen | time to reach | of nitrogen | time to reach |
| pretreatment | compound | max. recovery | compound | max. recovery |
| medium | (wt. %) | (min) | (wt. %) | (min) |
| Butylamine | 99.5 | 210 | 94.4 | 350 |
| Triethylammonium | 98.5 | 280 | 60.8 | 450 |
| formate | | | | |

In the method with this mechanical treatment step higher maximum recovery rates are reached in a faster time. These results impressively show the effect of the plurality of blades (kneaders) arranged to rotate within the vessel, which ensure that the mixture is treated such that it is exposed to shearing and forced to form a continuously renewed evaporation surface between the mixture and a gaseous phase.

### Example 2B: Evaluation of the Mechanical Processing on Recovery of a Pretreatment Medium Comprising an Ionic Liquid and a Co-Solvent

In analogy to the process in Example 2A a further biomass material was provided in a mixture with a medium comprising the ionic liquid pyridinium formate and the co-solvent water.

The biomass mixture containing sawdust from spruce wood was prepared by the following pretreatment process: Spruce wood sawdust was dried for 24 h at 40° C. and sieved to a grain size of ≤2 mm. 1 kg of the dry material was transferred into a 20 L pressure tight stainless steel autoclave (Kiloclave, Buechi AG) and combined with 5.7 kg of the pretreatment medium comprising 70 wt % of the ionic liquid pyridinium formate (CAS 15066-28-1, T_{b}=113° C. @ 1 atm, ID 5 in Table 1) and 30 wt % water. The autoclave was equipped with an impeller stirrer and heated for 2 hours at 80° C. under stirring at 100 rpm. After this treatment the mixture showed a pasty consistence and a dark brown color.

The mixture was independently feed into a) conventional rotary evaporator (Heidolph Instruments GmbH & CO. KG, Schwabach, Germany) and b) a laboratory model of a large-volume processor (Werner & Pfleiderer Lebensmitteltechnik GesmbH, formerly Werner & Pfleiderer A G, Vienna, Austria) as described in Example 2A.

Both apparatuses were operated with a rotational speed of 50 rpm and a pressure of 100 mbar (vacuum) was applied in both setups and the vessels were heated to a temperature of 100° C.

In the conventional rotary evaporator, recovery of the pretreatment medium was determined as a function of time by the mass loss of the mixture. In the lab-scale large-volume processor, the actual recovery of the pretreatment media was determined as it was condensed downstream using a cold trap operated with liquid nitrogen.

The results for the recovery of the pretreatment medium comprising the ionic liquid pyrimidium formate and water are shown in FIG. 3B, for both conventional rotary evaporator (triangles) and a laboratory model of a large-volume processor (squares). The recovery rates at certain time points are also indicated in Table 3 below.

TABLE 3
Recovery of the pretreatment medium comprising pyrimidium
formate and water using different processing apparatuses

| | lab scale large- | conventional |
|---|---|---|
| | volume processor | rotary evaporator |
| time | recovery of nitrogen | recovery of nitrogen |
| (min) | compound (wt. %) | compound (wt. %) |
| 0 | 0.00 | 0.0 |
| 15 | 14.5 | 11.3 |
| 30 | 31.6 | 20.2 |
| 60 | 64.6 | 26.4 |
| 90 | 83.5 | 31.3 |
| 120 | 91.0 | 35.4 |
| 150 | 96.2 | 41.5 |
| 180 | 98.2 | 46.0 |
| 240 | 98.7 | n.d. |
| 360 | 98.7 | 54.6 |
| 400 | n.d. | 56.2 |

Abbreviation: n.d., not determined

The data clearly confirm the role of the mechanical treatment step as higher maximum recovery rates are reached in a faster time in the lab scale large-volume processor compared to the conventional rotary evaporator, which applies similar conditions without a plurality of blades treating the mixture such that it is exposed to shearing and forced to form a continuously renewed evaporation surface between the mixture and a gaseous phase.

### Example 3: Description of Suitable Processors

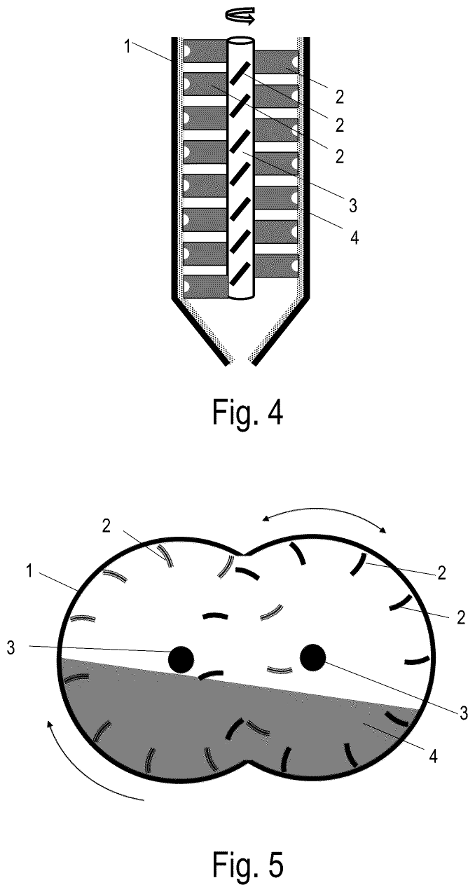

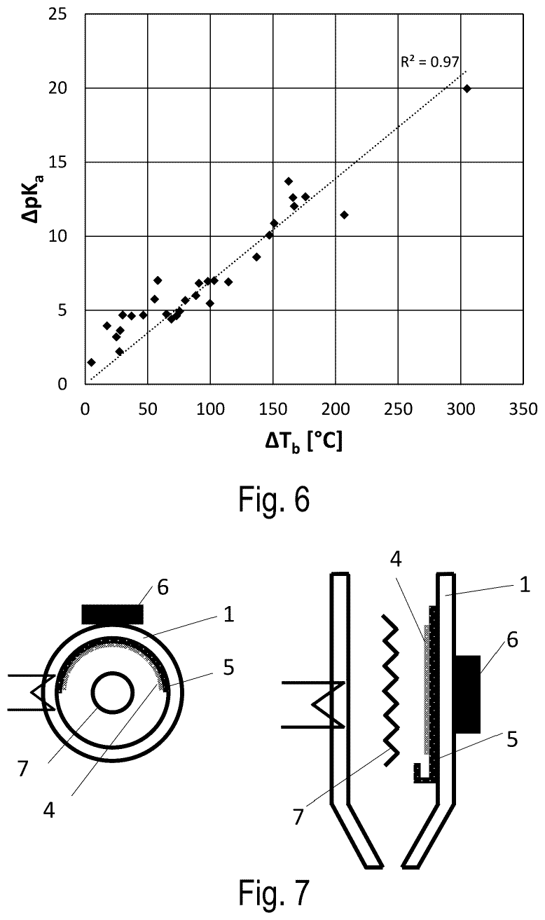

An exemplary embodiment of an apparatus being a thin-film processor, wherein the rotating plurality of blades defines one rotating axis, and the vessel essentially is a cylinder with a vertical orientation, is schematically shown in FIG. 4.

FIG. 4 is a scheme of thin-film processor, wherein the vessel 1 is shown without front to reveal the inside of the vessel 1. The apparatus has an essentially cylindric vessel 1 with a circular base. The vessel 1 can be filled with a load, i.e., a raw biomass material in mixture with a pretreatment medium, at the top of the vessel (not shown in FIG. 4).

Within the vessel 1, several linear sets of blades 2 are arranged on a central rotor element 3. If the central rotor element 3 rotates, the plurality of blades 2 rotates within the vessel along one rotational axis as shown with the arrow in FIG. 4.

Each of the blade extends radially to the internal surface of the vessel 1, leaving only a small radial clearance between the plurality of blades 2 and the internal surface of the vessel 1. Due to the staggered arrangement of the lines of blades 2, the plurality of blades defines a body of revolution essentially corresponding to the cylindric part of the vessel 1. When rotating, the plurality of blades 2 shear the load 4, e.g., the mixture, comprising the biomass material and a (pre) treatment medium, such that is spread out as a film. The figure depicts a state, wherein the mixture 4 is distributed uniformly as a film at the internal surface of the vessel 1.

The blades 2 are bent with respect to the rotational axis, such that they transport the load continuously. This allows for mass transport along the rotational axis 3, i.e., towards the lower part of the cylindric vessel 1, wherein the step of collecting the remaining biomass can be performed. Additionally, the movement along the rotational axis 3 also ensures renewal of the surface of evaporation as the film 4 is continuously rebuilt.

The nitrogen compound evaporates from the mixture and moves into the gaseous phase, wherein it can further pass through the vessel. The plurality of blades 2 is arranged to allow a gaseous stream. The gaseous stream may be removed from the vessel by means of evacuation (not shown), preferably arranged to retrieve a gaseous stream, e.g., at the top of the vessel.

Another exemplary embodiment of an apparatus, i.e., a large-volume processor, is exemplarily shown in FIG. 5.

FIG. 5 shows a sectional view through the vessel 1 of the apparatus and reveals how a plurality of blades 2 is arranged to rotate around two rotating axes 3.

The vessel 1 has the form of a cylinder with an essentially bicircular base area and is operated in horizontal orientation. Accordingly, the load 4, i.e., the mixture, comprising the biomass material and a (pre) treatment medium, accumulates at the lower part of the vessel 1. However, as the blades 2 rotate around the rotational axes 3 (as indicated by the arrows in FIG. 5), they expose the mixture 4 to shearing and force it to form a continuously renewed evaporation surface between the mixture 4 and a gaseous phase in the vessel.

As the plurality of blades 2 are configured as kneaders, the sectional view does not show how each of the blades is connected to one of the rotor elements 3. The open arrangement of the blades allows for a continuous, large gaseous phase within the vessel. Furthermore, the plurality of blades 2 arranged on one rotor axis 3 interacts with the other rotor axis 3 in a way that ensures full circulation of the biomass mixture in radial and axial direction and self-cleaning of the apparatus, preventing the formation of any pools of biomass.

The vessel as shown in FIG. 5 has no means for transportation of the mixture/biomass into or from the vessel 1. Thus, the mixture is loaded/feed into the vessel 1 batch-wise.

### Example 4: Implementation of the Method According to the Invention in a Process for Production of Biofuels and Comparison with Standard Processes

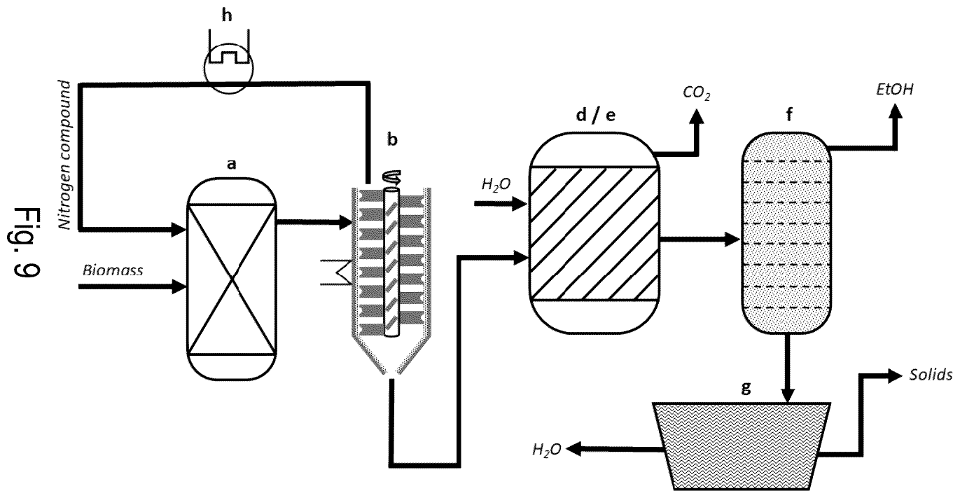

In order two visualize how the method according to the invention can be implemented in a process for production of ethanol as biofuel, the FIGS. 8 and 9 show a comparison of a process according to the art (FIG. 8) and the process based on the method according to the invention (FIG. 9).

FIG. 8 shows a state of the art process for using microbial production of advanced biofuels as recently summarized for example by Keasling et al. (Keasling, J., Garcia Martin, H., Lee, T. S. et al. Nat Rev Microbiol 19, 701-715 (2021). DOI: 10.1038/s41579-021-00577-w). The biomass and the pretreatment medium (e.g., the nitrogen compound) are mixed in a first reactor for pretreatment (a). Subsequently, the washing step (c) involves washing with water for several runs (x-runs) to remove the nitrogen compound and soluble lignin as well as the washing water and impurities. The remaining biomass is further subjected to (enzymatic) hydrolysis (d) to liberate the sugars from the biomass material, which step is typically performed in aqueous medium. Before hydrolysis, typically the pH must be adjusted with acids to neutral or slightly acidic pH value. This is the case as though washing off the pretreatment medium the alkaline pH remains and would hinder effective hydrolysis. Subsequently, fermentation (e) of the sugars to ethanol and CO₂ is also performed in aqueous medium. The final step of the ethanol production is distillation (f). To recover the water and the ionic liquid a complex system involving solid liquid separation (g) and ultrafiltration (i) is required to first remove the solid fractions and subsequently, the nitrogen compound may be recovered from water by distillative removal of the water (j). Even if the water is subjected to waste-water-treatment and/or regenerated for subsequent use in the process, the overall process obviously requires cumbersome and energy intensive steps for recovery of the (pre) treatment agent.

In comparison, FIG. 9 impressively shows that the simplification of the process, when relying on a method according to the invention, i.e., when separating/recovering the pretreatment medium comprising a nitrogen compound is achieved according to the invention. The distillable pretreatment medium is fully separated in this process step (b), herein visualized by a thin-film apparatus, which can be operated continuously. No washing step (c) is required. As the remaining biomass is obtained essentially pure and dry, it can be directly transferred in an aqueous medium, which allows for performing both hydrolysis (d) and fermentation (e) in a one pot setup. Of note, the lignins and impurities remaining in the biomass have no negative influence on hydrolysis and fermentation of the sugars of the biomass. Subsequent separation of the formed product ethanol by distillation (f) from the lignin, residuals and impurities and solid-liquid-separation (g) can be performed according to the state of the art, but with significantly lower water content.

The nitrogen compound is recovered in a condensation step (h), at a condenser outside of the apparatus for the separation (b), and directly recovered for re-use as pretreatment medium.

1. A method for processing a biomass material, wherein the biomass material is provided in the form of a mixture, the mixture comprising the biomass material and a medium comprising a nitrogen compound, in particular a treatment and/or pretreatment medium, and wherein said method comprises the steps of:
feeding the mixture into a vessel of an apparatus, wherein the apparatus comprises means to heat an internal surface of the vessel, and a plurality of blades arranged to rotate within the vessel,
treating the mixture with the plurality of blades such that the mixture is exposed to shearing, and forced to form a continuously renewed evaporation surface between the mixture and a gaseous phase,
allowing the nitrogen compound to form a gaseous form and allowing the gaseous form to pass through the evaporation surface and move into the gaseous phase,
removing the gaseous phase comprising the nitrogen compound in gaseous form from the vessel, and
collecting the remaining biomass material.

2. The method according to claim 1, further including recovering of the medium, wherein the nitrogen compound is distillable, the nitrogen compound is allowed to evaporate in the gaseous form, and the method further comprises a step of
condensing the nitrogen compound out of the gaseous phase removed from the vessel, such that at least a part of the medium is recovered.

3. The method according to claim 1, wherein the rotating plurality of blades defines at least one rotational axis.

4. The method according to claim 1, wherein the rotating plurality of blades has a radial clearance to the internal surface of the vessel in the range of from 0.1 to 50 mm, preferably from 0.1 to 20 mm, such as from 0.5 to 5 mm or from 1 to 10 mm.

5. The method according to claim 1, wherein the vessel has essentially the geometric form of a right cylinder with a base area having either the form of a circle or overlapping circles.

6. The method according to claim 1, wherein the apparatus is selected from the group consisting of
thin-film processors, wherein the rotating plurality of blades defines one rotating axis, and the vessel essentially is a cylinder with a vertical orientation, and
processors, wherein the rotating plurality of blades defines one or two rotating axes, and the vessel essentially is a cylinder with a horizontal orientation, in particular large-volume processors.

7. The method according to claim 1, wherein the plurality of blades rotates with a maximal peripheral speed in the range of from 0.2 to 5 m/s, preferably, from 0.5 to 2 m/s, such as about 1 m/s.

8. The method according to claim 1, wherein the gaseous phase is removed with a gas stream, wherein the gas stream is withdrawn from the vessel by means of evacuation and/or a guided strip gas stream, preferably evacuation.

9. The method according to claim 1, wherein the method is performed at a pressure in the range of from 10⁻² mbar to 10³ mbar, preferably the range of from 10⁻¹ mbar to 100 mbar, more preferably in the range of from 0.5 to 10 mbar, such as 1 mbar.

10. The method according to claim 1, wherein the internal surface is heated to a temperature in the range of from 20° C. to 200° C., preferably of from 50° C. to 170° C., more preferably of from 80° C. to 160° C., such as from 100 to 140° C.

11. The method according to claim 1, wherein the mixture has an initial shear viscosity of from 10 to 50,000 Pa*s, preferably in the range of from 100 to 20,000 Pa*s, more preferably about 500 to 15,000 Pa*s, wherein the shear viscosity of the initial mixtures preferably is determined using an oscillation rheometer with a two plates geometry at a shear rate of 1 s⁻¹ and a measuring temperature of about 25° C.

12. The method according to claim 1, wherein the mixture has an initial content of medium in the range of from 20 to 90 wt. %.

13. The method according to claim 1, the nitrogen compound is a protonatable or a protonated nitrogen compound.

14. The method according to claim 1, wherein the nitrogen compound is selected from the group consisting of
neutral nitrogen compounds,
nitrogen-based protic ionic liquids, preferably an ionic liquid selected from the group of acid base conjugate salt ionic liquids and alkyl carbamate ionic liquids,
and hybrid forms thereof.

15. The method according to claim 1, wherein the medium has content of the nitrogen compound in the range of from 10 to 100 wt. %.

16. The method according to claim 12, wherein the mixture has an initial content of medium in the range of from 30 to 95 wt. %.

17. The method according to claim 12, wherein the mixture has an initial content of medium in the range of from 50 to 90 wt. %.

18. The method according to claim 12, wherein the mixture has an initial content of medium in the range of from 60 to 85 wt. %.

19. The method according to claim 15, wherein the medium has content of the nitrogen compound in the range of from 50 to 100 wt. %.

20. The method according to claim 15, wherein the medium has content of the nitrogen compound of essentially 100 wt. %.
